(19) United States
(12) Patent Application Publication
(10) Pub. No.: US 2002/0164633 A1
(43) Pub. Date: Nov. 7, 2002

(54) Cell-based assay for detecting activation of poly (ADP-ribose) polymerase

(75) Inventors: Csaba Szabo, Gloucester, MA (US); Laszlo Virag, Lugossy (HU)

Correspondence Address: PENNIE AND EDMONDS, 1155 AVENUE OF THE AMERICAS, NEW YORK, NY, 100362711

(73) Assignee: Inotek Pharmaceuticals Corporation

(21) Appl. No.: 10/125,326

(22) Filed: Apr. 18, 2002

Related U.S. Application Data
(60) Provisional application No. 60/285,175, filed on Apr. 20, 2001.

(51) Int. Cl. C12Q 1/68; G01N 33/567; G01N 33/53

(52) U.S. Cl. 435/6; 435/7.21; 435/7.5

(57) ABSTRACT

The present invention relates to methods of detecting poly(ADP-ribose) polymerase (“PARP”) activity in cells and tissue by contacting a cell with an effective amount of biotinylated NAD⁺ and detecting the presence of incorporated biotin within the cell. The invention also relates to the identification oxidatively stressed tissues and cells by detecting PARP activity. The invention also relates to identifying a patient suffering from a disorder that causes PARP overexpression.

This application claims the benefit of U.S. Provisional Application No. 60/285,175 filed Apr. 20, 2001, which is incorporated herein by reference in its entirety.

### FIELD OF THE INVENTION

The present invention relates to methods of detecting poly(ADP-ribose) polymerase (“PARP”) activity in cells and tissue by contacting a cell with an effective amount of biotinylated NAD⁺ and detecting the presence of incorporated biotin within the cell. The invention also relates to the identification oxidatively stressed tissues and cells by detecting PARP activity. The invention also relates to identifying a patient suffering from a disorder that causes PARP overexpression.

### BACKGROUND OF THE INVENTION

Poly(ADP-ribose) polymerase (“PARP”) is a nuclear enzyme that becomes activated in response to DNA damage (de Murcia & Menissier de Murcia, 1994, Trends Biochem Sci 19:172-176). Activated PARP cleaves NAD to nicotinamide and ADP-ribose, and polymerizes the latter on to nuclear acceptor proteins such as histones, transcription factors and PARP itself. Poly-ADP ribosylation contributes to DNA repair and to the maintenance of genomic stability (Wang et al., 1997, Genes Dev 11:2347-58; Simbulan-Rosenthal et al., 1999, Simbulan-Rosenthal et al 1999, Proc. Natl. Acad. Sci. U.S.A. 96:13191-13196; and Muiras & Bürkle, 2000, Exp. Gerontol. 35:703-709). During inflammation, ischemia-reperfusion or shock, free radical/oxidant-induced DNA single strand breakage triggers the over-activation of PARP, which leads to depletion of NAD (Szabo & Dawson 1998, Trends Pharmacol. Sci. 19:287-298 and Szabó, 2000, Cell Death: the Role of PARP. Boca Raton, Fla., CRC Press). In an effort to re-synthesize NAD+, ATP will also be consumed, resulting in necrotic type cell death (Schraufstatter et al., 1986, Proc. Natl. Acad. Sci. U.S.A. 83:4908-4912; Virag et al., 1998, Immunology 94:345-355 and Virag et al., 1998, J. Immunol. 161:3753-3759). This PARP mediated suicidal pathway has been implicated in the cell death of immune stimulated macrophages, as well as in peroxynitrite- or hydrogen peroxide-induced dysfunction or cell death of thymocytes, macrophages, endothelial cells, neuronal cells and fibroblasts (Soriano et al., 2001, Nat Med 7:108-13; Virag et al., 1998, Immunology 94:345-355; Szabóet al., 1998, Proc Natl Acad Sci U.S.A. 95:3867-3872; and Zingarelli et al., 1996, J. Immunol. 156:350-358). Inhibition of PARP activity by pharmacological inhibitors or the absence of functional PARP enzyme in PARP knock out animals provides significant protection in animal models of a wide variety of diseases including various forms of inflammation, shock, stroke, myocardial ischemia, diabetes and diabetic endothelial dysfunction (Szabo & Dawson 1998, Trends Pharmacol. Sci. 19:287-298 and Szabó, 2000, Cell Death: the Role of PARP. Boca Raton, Fla., CRC Press).

PARP activity is typically measured by the incorporation of radioactivity from isotope-labeled NAD⁺ into TCA precipitable proteins (Schraufstatter et al., 1986, Proc. Natl. Acad. Sci. U.S.A. 83:4908-4912). Alternatively, poly(ADP-ribose), the product of the PARP catalyzed reaction can be purified from cells and tissues by a tedious procedure, and the polymer can be quantified by HPLC (Kiehlbauch et al., 1993, Anal Biochem 208:26-34 and Shah et al., 1995 Anal Biochem 227:1-13). A more convenient approach for the assessment of PARP activation is the detection of poly(ADP-ribose) by a monoclonal antipoly(ADP-ribose) antibody in western blots, dot blots, immunocytochemistry, and flow cytometry (Affar et al., 1998, Anal Biochem 259:280-3 and Affar et al., 1999, Biochim Biophys Acta 1428:137-46). However, the use of the murine monoclonal anti-poly(ADPribose) antibody in mouse tissues especially in inflamed tissues often results in high background staining (Virag L, unpublished observations). Moreover, the amount of the polymer synthesized does not necessarily reflect the degree of PARP activation, as poly(ADP-ribose) in cells and tissues is rapidly metabolized by poly(ADP-ribose) glycohydrolase (PARG) (Ueda et al., 1972, Biochem Biophys Res Commun 46:516-23).

Citation or identification of any reference in the background section of this application is not an admission that such reference is available as prior art to the present invention.

### SUMMARY OF THE INVENTION

The invention is based, in part, on the discovery of a non-radioactive method for assaying the activity of PARP in in situ, i.e., cell-based systems.

In a first embodiment, the invention provides a method for identifying a poly(ADP-ribose) polymerase-expressing cell by contacting a cell with an effective amount of biotinylated NAD⁺ and detecting the presence of incorporated biotin with the cell.

In a second embodiment, the invention provides a method for identifying a poly(ADP-ribose) polymerase-expressing tissue by contacting a tissue with an effective amount of biotinylated NA⁺ and detecting the presence of incorporated biotin with the tissue.

In a third embodiment, the invention provides a method for determining the amount of poly(ADP-ribose) polymerase in a cell or tissue by contacting a poly(ADP-ribose) polymerase-expressing cell or tissue with an effective amount of biotinylated NAD⁺ and determining the amount of incorporated biotin within a cell.

In a fourth embodiment, the invention provides a method for identifying inflamed tissue by contacting a cell or tissue of the patient with an effective amount of biotinylated NAD⁺ and detecting the presence of incorporated biotin within the cell or tissue.

In a fifth embodiment, the invention provides a method for identifying a patient with an oxidative stress disorder by contacting a cell or tissue of the patient with an effective amount of biotinylated NAD+ and detecting the presence of incorporated biotin within the cell or tissue.

In a sixth embodiment, the invention provides a method for identifying a patient with inflammation, shock, repurfusion injury, stroke, myocardial ischemia, diabetes, diabetic endothelial dysfunction, atherogenesis, and atherosclerosis by contacting a cell or tissue of the patient with an effective amount of biotinylated NAD⁺ and detecting the presence of incorporated biotin within the cell or tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1D are images showing basal and hydrogen peroxide-induced polyADP ribosylation in J774 macrophages. Untreated (FIGS. 1A-B) and hydrogen peroxide-treated J774 cells were stained with the bio-NAD method, as described in Materials and Methods. A low intensity nuclear staining could be visualized with the assay in untreated cells reflecting baseline PARP activity (FIGS. 1A-B). Mitotic cells displayed increased poly-ADP-ribosylating activity (arrows). In response to hydrogen peroxide (500 μM) a markedly increased nuclear staining could be detected in J774 cells (FIG. 1C) but not in cells pretreated with the PARP inhibitor PJ34 (5 μM) (FIG. 1D).

FIGS. 2A-2C are images showing enzyme histochemical detection of PARP activation in hydrogen peroxide-treated skin. Frozen sections were cut from untreated (FIG. 2A) and hydrogen peroxide-treated (FIGS. 2B-C) mouse skin and were stained with the bio-NAD method. Panel C shows a section stained in the presence of the PARP inhibitor 3 aminobenzamide (5 mM).

FIG. 3 is a histogram showing detection of hydrogen peroxide-induced PARP activation in J774 cells with the PARP-CELISA method. J774 cells were seeded in 96 well plates and were treated with the indicated concentration of hydrogen peroxide in the absence or presence of 5 μM PJ34. PARP activity was detected with the CELISA method as described in the Examples.

### DETAILED DESCRIPTION OF THE INVENTION

Poly(ADP-ribose) polymerase is a nuclear enzyme activated by DNA damage. Activated PARP cleaves NAD⁺ into nicotinamide and (ADP-ribose) and polymerizes the latter on nuclear acceptor proteins. PARP is involved in the maintenance of genomic integrity. However, over-activation of PARP by reactive oxygen and nitrogen intermediates represents a pathogenetic factor in various forms of inflammation, shock and reperfusion injury. Using the substrate 6-biotin-17-nicotinamide-adenine-dinucleotide (bio NAD⁺) Applicants have developed three in situ applications, including cytochemistry, enzyme histochemistry and cellular ELISA, in order to detect the activation of PARP in oxidatively stressed cells and tissues. With the novel assay one is able to detect basal and hydrogen peroxide-induced PARP activity in J774 macrophages. The method can be used for the detection of PARP activity in cells and tissues. Also provided is a cellular ELISA (CELISA) assay to quantify PARP activation in cultured cells, and an enzyme cytochemical/histochemical reaction to detect PARP activation in oxidatively stressed cells and tissues.

Applicants have found that 6-biotin-17-nicotinamide-adenine-dinucleotide (bio NAD⁺) (Zhang & Snyder, 1993, Biochemistry 32:2228-2233) can be used as a substrate of PARP in cells and tissues. In untreated J774 macrophage cells, bio-NAD+ metabolizing activity is detected in the nuclei. The nuclear staining pattern indicates that bio-NAD⁺ is metabolized most likely by PARP a nuclear enzyme polymerizing ADP-ribose units on nuclear acceptor proteins. It has been shown previously that bio-NAD⁺ can also serve as substrate for mono-ADP ribosylation in vitro (Zhang & Snyder, 1993, Biochemistry 32:2228-2233). However, this G protein coupled process takes place in the plasma membrane. Therefore, mono-ADP-ribosylation is not likely to contribute to bio-NAD+ metabolism in Applicants' system. Applicants have observed an interesting phenomenon in untreated J774 metabolism in Applicants' system. Applicants have observed an interesting phenomenon in untreated J774 cells: mitotic cells displayed strong nuclear positivity as compared to interphase cells. Numerous cells representing various stages of mitoses could be found with strong reactivity localized to the condensed chromatin but not to the interchromatin areas. This observation, however, is not surprising in light of previous observations linking PARP to the process of replication. PARP has been shown to associate with and regulate the activity of topoisomerase I an enzyme uncoiling DNA by temporarily cutting into one of the DNA strands (Ferro et al., 1984, Adv Exp Med Biol 179:441-7; Bauer et al., 2000, Int. J. Mol. Med. 5:533-540; Bauer & Kun, 2000, Int. J. Mol. Med. 6:153-154). Association of PARP with other proteins involved in replication such as the DNA polymerase I-primase complex has also been demonstrated (Dantzer et al., 1998, Nucleic Acids Res 26:1891-8). Furthermore, it has also been reported that in PARP knock out cells but not in cells treated with PARP inhibitors, a tetraploid population emerges indicating that the presence of PARP protein but not PARP activity is required to maintain chromosomal stability (Simbulan-Rosenthal et al 1999, Proc. Natl. Acad. Sci. U.S.A. 96:13191-13196 and Simbulan-Rosenthal et al., 2001, Nucleic Acids Res. 29:841-849). Although PARP protein has previously been shown to localize to centrosomes and chromosomes during cell division and in the interphase (Kanai et al., 2000, Biochem. Biophys. Res. Commun. 278:385-389), to Applicants' best knowledge, this is the first morphological demonstration of increased poly(ADP-ribose) polymerase activity in mitotic cells.

In line with previous data reporting PARP activation in oxidatively stressed cells (Virag et al., 1998, Immunology 94:345-355 and Szabóet al., 1998, Proc Natl Acad Sci U.S.A. 95:3867-3872), treatment of J774 cells with hydrogen peroxide caused a marked enhancement of nuclear bio-NAD⁺ staining. Hydrogen peroxide-induced bio-NAD⁺ metabolism could be blocked by 3-aminobenzamide, an inhibitor of PARP, providing further support that PARP activity is responsible for bio-NAD⁺ staining in Applicants' system. The question arises as to what degree the different PARP isoenzymes contribute to bio-NAD⁺ incorporation under basal conditions and after oxidative stress. Until recently, PARP activity was believed to result from the function of a single enzyme. After the observation that PARP-deficient cells have some residual PARP activity (Shieh et al., 1998, J. Biol. Chem. 273:30069-30072), intensive research began to identify enzymes responsible for this activity. In the past few years, four other enzymes possessing poly(ADP-ribosylation) activity have been described (for review, see Szabó, 2000, Cell Death: the Role of PARP. Boca Raton, Fla., CRC Press) and named PARP 2-5, with the founding member of the PARP enzyme family now designated as PARP-1. Although research on the biological role of these novel PARP enzymes is in the embryonal stage, interesting differences in domain structure, subcellular localization, tissue distribution, and ability to bind to DNA have already been established. Applicants' data showing increased bio-NAD⁺ incorporation in wild-type but not in PARP-1 deficient macrophages indicates that, at least under conditions of oxidative stress, PARP-1 is responsible for bio-NAD⁺-incorporating activity (data not shown).

In situ immunohistochemical demonstration of PARP activation in tissues by detecting poly(ADP-ribose) was successfully demonstrated in some cases by Applicants (Liaudet et al., 2000, Proc. Natl. Acad. Sci. U.S.A. 97:10203-10208) and by others (Eliasson et al., 1997, Nat. Med. 3:1089-1095). However, in mouse tissues, evaluation of stainings obtained by using the mouse monoclonal anti-poly(ADP-ribose) antibody is often difficult due to possible cross-reaction of the secondary anti-mouse immunoglobulin-peroxidase conjugates with the endogenous immunoglobulins that are present in the mouse tissues. This difficulty may be very difficult to overcome in inflamed tissues where extravasation leads to increased immunoglobulin content. This technical problem can be circumvented by the use of bio-NAD⁺ enzyme histochemistry. Using this assay, PARP activation has been detected and localized in vascular tissues of diabetic mice in Applicants' recent studies (Soriano et al., 2001, Nat Med 7:108-13).

Another technical difficulty that needs to be considered is the removal of poly(ADPribose) by poly(ADP-ribose) glycohydrolase (PARG). This could theoretically also occur with biotinyl-poly(ADP-ribose). Bürkle et al has previously reported that PARG can be inactivated by treating cells with trichloroacetic acid (Lankenau et al., 1999, Chromosoma 108(1):44-51). Therefore, Applicants have included, this step into the cytochemical procedure in order to prevent removal of the incorporated biotinylated-ADP-ribose. In tissues, however, Applicants found that TCA treatment was unnecessary.

For a long time the only assay in which PARP inhibitors could be tested was a radioactive method using either ³²p or ³H-labeled NAD⁺. This assay could be used both with purified PARP and also in cellular systems. A bio-NAD⁺ assay provides a non-radioactive alternative to assess the efficacy of potential PARP inhibitors in a cell free system. The CELISA protocol has now extended the applicability of the bio-NAD⁺ substrate to the measurement of cellular PARP activity. It is important to emphasize here that according to Applicants' experience, due to differences in cell permeability of different compounds the IC₅₀ values may dramatically differ in a cellular PARP assay, as compared to cell-free assays. Therefore Applicants believe that elaboration of a CELISA method for the quantitation of cellular PARP activity represents an important advancement aiding more successful research in the PARP field.

In summary, Applicants have developed three applications to detect or to measure PARP activation in cells and tissues. The assays are based on the use of biotinylated-NAD⁺ as a PARP substrate. The straightforward protocols described herein allow a simple, cost effective two-step detection of activated PARP in oxidatively stressed tissues and cells.

In a first embodiment, the invention provides a method for identifying a poly(ADP-ribose) polymerase-expressing cell by contacting a cell with an effective amount of biotinylated NAD' and detecting the presence of incorporated biotin with the cell. Contacting a cell with biotinylated NAD⁺ is in situ or in vivo. In a preferred embodiment, the incorporated biotin is detected with a conjugated streptavidin detection system such as, but not limited to, streptavidin peroxidase or peroxidase-conjugated streptavidin. In a preferred embodiment, the method further comprises fixing the cell with trichloracetic acid (TCA). The cells include, but are not limited to, mitotic cells, oxidatively stressed cells, macrophages, thymocytes, endothelial cells, neuronal cells, or fibroblasts.

In a second embodiment, the invention provides a method for identifying a poly(ADP-ribose) polymerase-expressing tissue by contacting a tissue with an effective amount of biotinylated NAD⁺ and detecting the presence of incorporated biotin with the tissue. Contacting a tissue with biotinylated NAD⁺ is in situ or in vivo. In a preferred embodiment, the incorporated biotin is detected with a conjugated streptavidin detection system such as, but not limited to, streptavidin peroxidase or peroxidase-conjugated streptavidin. In a preferred embodiment, the tissue comprises cells including, but not limited to, mitotic cells, oxidatively stressed cells, macrophages, thymocytes, endothelial cells, neuronal cells, or fibroblasts. In another preferred embodiment, the tissue includes, but is not limited to, skin and vascular tissue.

In a preferred embodiment, the presence of incorporated biotin within a tissue is determined comprising (a) detecting incorporated biotin with a conjugated streptavidin detection system, such as but not limited to, streptavidin peroxidase or peroxidase-conjugated streptavidin; (b) developing a reaction of said conjugated streptavidin with said incorporated biotin with a calorimetric substrate; and (c) counterstaining said incorporated biotin reacted with said calorimetric substrate. In a preferred embodiment, the calorimetric substrate is cobalt-enhanced nickel-DAB substrate and the counterstain is Nuclear fast Red.

In a third embodiment, the invention provides a method for determining the amount of poly(ADP-ribose) polymerase in a cell by contacting a poly(ADP-ribose) polymerase-expressing cell with an effective amount of biotinylated NAD⁺ and determining the amount of incorporated biotin within a cell. Contacting a cell with biotinylated NAD⁺ is in situ or in vivo. In a preferred embodiment, the incorporated biotin is detected with a conjugated streptavidin detection system such as, but not limited to, streptavidin peroxidase or peroxidase-conjugated streptavidin. In a preferred embodiment, the method further comprises fixing the cell with trichloracetic acid (TCA). The cells include, but are not limited to, mitotic cells, oxidatively stressed cells, macrophages, thymocytes, endothelial cells, neuronal cells, or fibroblasts. In a preferred embodiment, the amount of incorporated biotin within a cell is measured comprising (a) detecting incorporated biotin with a conjugated streptavidin detection system; (b) developing a reaction of said conjugated streptavidin with said incorporated biotin with a calorimetric substrate; and (c) measuring optical density of said colorimetric substrate with a spectrophotometer. In a preferred embodiment, the calorimetric substrate is TACS-Sapphire™.

In a fourth embodiment, the invention provides a method for identifying inflamed tissue by contacting a cell or tissue of the patient with an effective amount of biotinylated NAD⁺ and detecting the presence of incorporated biotin within the cell or tissue. Contacting a cell or tissue with biotinylated NAD⁺ is in situ or in vivo. The invention also provides a method for assessing treatment in a patient having inflamed tissue by administering an effective amount of biotinylated NAD⁺ and detecting the presence of incorporated biotin within the cell or tissue, i.e., PARP is used as a marker of inflamed tissue. In a preferred embodiment, the incorporated biotin is detected with a conjugated streptavidin detection system such as, but not limited to, streptavidin peroxidase or peroxidase-conjugated streptavidin. In a preferred embodiment, the method further comprises fixing the cell with trichloracetic acid (TCA). The cells include, but are not limited to, mitotic cells, oxidatively stressed cells, macrophages, thymocytes, endothelial cells, neuronal cells, or fibroblasts. In a preferred embodiment, the tissue comprises cells including, but not limited to, mitotic cells, oxidatively stressed cells, macrophages, thymocytes, endothelial cells, neuronal cells, or fibroblasts. In another preferred embodiment, the tissue includes, but is not limited to, skin and vascular tissue.

In a preferred embodiment, the presence of incorporated biotin within a tissue is determined comprising (a) detecting incorporated biotin with a conjugated streptavidin detection system, such as but not limited to, streptavidin peroxidase or peroxidase-conjugated streptavidin; (b) developing a reaction of said conjugated streptavidin with said incorporated biotin with a calorimetric substrate; and (c) counterstaining said incorporated biotin reacted with said colorimetric substrate. In a preferred embodiment, the calorimetric substrate is cobalt-enhanced nickel-DAB substrate and the counterstain is Nuclear fast Red.

In a fifth embodiment, the invention provides a method for identifying a patient with an oxidative stress disorder by contacting a cell or tissue of the patient with an effective amount of biotinylated NAD⁺ and detecting the presence of incorporated biotin within the cell or tissue. Contacting a cell or tissue with biotinylated NAD⁺ is in situ or in vivo. The invention also provides a method for assessing treatment in a patient with an oxidative stress disorder by administering an effective amount of biotinylated NAD⁺ and detecting the presence of incorporated biotin within the cell or tissue, i.e., PARP is used as a marker of cells and/or tissues undergoing oxidative stress. In a preferred embodiment, the incorporated biotin is detected with a conjugated streptavidin detection system such as, but not limited to, streptavidin peroxidase or peroxidase-conjugated streptavidin. In a preferred embodiment, the method further comprises fixing the cell with trichloracetic acid (TCA). The cells include, but are not limited to, mitotic cells, oxidatively stressed cells, macrophages, thymocytes, endothelial cells, neuronal cells, or fibroblasts. In a preferred embodiment, the tissue comprises cells including, but not limited to, mitotic cells, oxidatively stressed cells, macrophages, thymocytes, endothelial cells, neuronal cells, or fibroblasts. In another preferred embodiment, the tissue includes, but is not limited to, skin and vascular tissue.

In a preferred embodiment, the presence of incorporated biotin within a tissue is determined comprising (a) detecting incorporated biotin with a conjugated streptavidin detection system, such as but not limited to, streptavidin peroxidase or peroxidase-conjugated streptavidin; (b) developing a reaction of said conjugated streptavidin with said incorporated biotin with a calorimetric substrate; and (c) counterstaining said incorporated biotin reacted with said colorimetric substrate. In a preferred embodiment, the calorimetric substrate is cobalt-enhanced nickel-DAB substrate and the counterstain is Nuclear fast Red.

Examples of clinical conditions in which oxidative species have been implicated include, but are not limited to, ischemia-reperfusion injury (e.g., stroke/myocardial infarction and organ transplantation), cancer, aging, arthritis associated with age, fatigue associated with age, alcoholism, red blood cell defects (e.g., favism, malaria, sickle cell anemia, Fanconi's anemia, and protoporphyrin photo-oxidation), iron overload (e.g., nutritional deficiencies, Kwashiorkor, thalassemia, dietary iron overload, idiopathic hemochromatosis), kidney (e.g., metal ion-mediated nephrotoxicity, aminoglycoside nephrotoxicity, and autoimmune nephrotic syndromes), gastrointestinal tract (e.g., oral iron poisoning, endotoxin liver injury, free fatty acid-induced pancreatitis, nonsteroidal antiinflammatory drug induced gastrointestinal tract lesions, and diabetogenic actions of alloxan), inflammatory-immune injury (e.g., rheumatoid arthritis, glomerulonephritis, autoimmune diseases, vasculitis, and hepatitis B virus), brain (e.g., Parkinson's disease, neurotoxins, allergic encephalomyelitis, potentiation of traumatic injury, hypertensive cerebrovascular injury, and vitamin E deficiency), heart and cardiovascular system (e.g., atherosclerosis, adriamycin cardiotoxicity, Keshan disease (selenium deficiency) and alcohol cardiomyopathy, eye (e.g, photic retinopathy, occular hemorrhage, cataractogenesis, and degenerative retinal damange), amyotrophic lateral sclerosis, and age-related macular degeneration (Slater, 1989, Free Rad. Res. Comm. 7:119-390; Deng et al., 1993, Science 261:1047-1051; Seddon et al., 1994, JAMA 272:1413-1420; Brown, 1995, Cell 80:687-692; and Jenner, 1991, ActaNeurol. Scand. 84:6-15).

In a sixth embodiment, the invention provides a method for identifying a patient with inflammation, shock, stroke, reperfusion injury, myocardial ischemia, diabetes, diabetic endothelial dysfunction, atherogenesis, and atherosclerosis by contacting a cell or tissue of the patient with an effective amount of biotinylated NAD⁺ and detecting the presence of incorporated biotin within the cell or tissue. Contacting a cell or tissue with biotinylated NAD⁺ is in situ or in vivo. In a preferred embodiment, the incorporated biotin is detected with a conjugated streptavidin detection system such as, but not limited to, streptavidin peroxidase or peroxidase-conjugated streptavidin. In a preferred embodiment, the method further comprises fixing the cell with trichloracetic acid (TCA). The cells include, but are not limited to, mitotic cells, oxidatively stressed cells, macrophages, thymocytes, endothelial cells, neuronal cells, or fibroblasts. In a preferred embodiment, the tissue comprises cells including, but not limited to, mitotic cells, oxidatively stressed cells, macrophages, thymocytes, endothelial cells, neuronal cells, or fibroblasts. In another preferred embodiment, the tissue includes, but is not limited to, skin and vascular tissue.

In a preferred embodiment, the presence of incorporated biotin within a tissue is determined comprising (a) detecting incorporated biotin with a conjugated streptavidin detection system, such as but not limited to, streptavidin peroxidase or peroxidase-conjugated streptavidin; (b) developing a reaction of said conjugated streptavidin with said incorporated biotin with a calorimetric substrate; and (c) counterstaining said incorporated biotin reacted with said colorimetric substrate. In a preferred embodiment, the colorimetric substrate is cobalt-enhanced nickel-DAB substrate and the counterstain is Nuclear fast Red.

The invention can be further illustrated in the following non-limiting examples.

### EXAMPLE 1

### Materials and Methods

Materials and methods used in the following examples are as follows. Biotinylated NAD⁺ and the TACS-Sapphire™ substrate were purchased from Trevigen (Gaithersburg, Md., USA). Streptavidin peroxidase was from Sigma (St. Louis, Mo.). Peroxynitrite was from Cayman Chemical Co. (Ann Arbor, Mich.). The PARP inhibitor PJ-34 was synthesized in the Applicants' laboratory, as described (Soriano et al., 2001, Nat Med 7:108-13). Cryoembedding medium was from Shandon (Pittsburgh, Penn.). Nuclear fast Red and Vectamount were obtained from Vector Laboratories (Burlingame, Calif.). All other chemicals were from Sigma (St. Louis, Mo.).

For application of hydrogen peroxide to the skin, hair was removed from the back of C57/BL6 mice (n=4) by Veet creme. Next day, hydrogen peroxide (250 nM /50 μl PBS pH7.4) was smeared onto the skin by a micropipette. Control animals were treated the same way with PBS. After 30 mm, mice were sacrificed and skin was excised. Samples were embedded in cryoembedding medium and immediately placed into a −70° C. freezer.

### EXAMPLE 2

### Cytochemical PARP Detection

J774. 1 cells were cultured on coverslips in RPMI medium supplemented with 10% FCS. 20 minutes after treatment with H₂O₂ (500 μM), the medium was removed and replaced with PARP reaction buffer (56 mM HEPES, 28 mM KCl, 28 mM NaCl, 2 mM MgCl₂, pH 8.0 complemented with 0.01% digitonin, 12.5 μM biotinylated NAD⁺ immediately before use). Control reactions were carried out in the presence of the PARP inhibitor 3-aminobenzarnide (5 mM). After 60 mm incubation at 37° C., the cells were fixed in 95% ethanol (10 mm at −20° C.) followed by 10 mm in 10% TCA (−20° C.). Coverslips were rinsed in PBS pH 7.4 (10 mm) and endogenous peroxidase was blocked by 0.5% H₂O₂/methanol for 15 minutes. After 2×5 min rinses with PBS 7.4, coverslips were blocked in 1% BSA/PBS for 30 minutes followed by two rinses in PBS-Triton X-100 (0.1%). Incorporated biotin was detected by streptavidin peroxidase (diluted 1:100 in PBS-Triton X-100 (30 mm at room temperature). Coverslips were washed (4×5 mm) with PBS pH7.4-Triton X-100 and color was developed with cobalt-enhanced nickel-DAB substrate. Coverslips were mounted with glycerol on slides and viewed with a Zeiss Axiolab microscope. Pictures were taken with a Zeiss Axiocam digital camera.

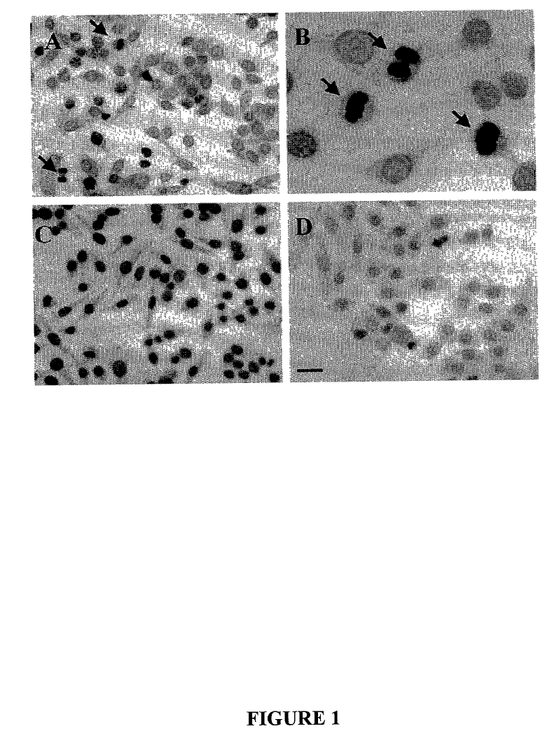

J774 macrophages were stained for PARP activity with the bio-NAD⁺ substrate (FIGS. 1A-1C). All cells showed a predominantly nuclear staining (FIG. 1A), indicating that the bioNAD⁺ metabolizing enzyme localizes in the nucleus. The intensity of nuclear staining in the vast majority of cells was moderate reflecting basal PARP activity. In sharp contrast to interphase cells, mitotic cells displayed an intense nuclear staining (FIGS. 1A, 1B). These cells appeared to be in the metaphase or ana-telophase of the mitotic cycle. Treatment of J774 cells with 500 μM H₂O₂ induced a marked enhancement of ADP-ribosylating activity, as indicated by the strong nuclear staining of H₂O₂-treated cells (FIG. 1C). Pretreatment of cells with the PARP inhibitor PJ34 (5 μM) 30 min prior to H₂O₂ exposure prevented enhancement of bioNAD⁺ staining (FIG. 1D). Similar results were also obtained with other types of cells, including fibroblasts and human keratinocytes.

### EXAMPLE 3

### PARP Enzyme Histochemistry

Cryosections (10 μm) were fixed for 10 mm in 95% ethanol at −20° C. and then rinsed 30 in PBS. Sections were permeabilized by 1% Triton X-100 in 100 mM TRIS pH 8.0, for 15 min. Reaction mixture (10 mM MgCl₂, 1 mM dithiothreitol, 30 μM biotinylated NAD⁺ , in 100 mM TRIS, pH 8.0) was then applied to the sections for 30 min at 37° C. Reaction mix containing 5 mM 3-aminobenzamide or biotinyl-NAD⁺ free reaction mix were used as controls. After three washes in PBS, incorporated biotin was detected by peroxidase-conjugated streptavidin (1:100, 30 min, room temperature). After 3×10 min washes in PBS, color was developed with cobalt-enhanced nickel-DAB solution (95 mg DAB, 1.6 NaCl, 2 g nickel sulfate and 25 μl of 30% hydrogen peroxide in 0.1 M acetate buffer pH 6.0) followed by 5 min incubation in TRIS-cobalt solution (1.2 g TRIS base, 1 g cobalt chloride in 200 ml distilled water, pH 7.2). Sections were counterstained in Nuclear fast Red, dehydrated and mounted in Vectamount.

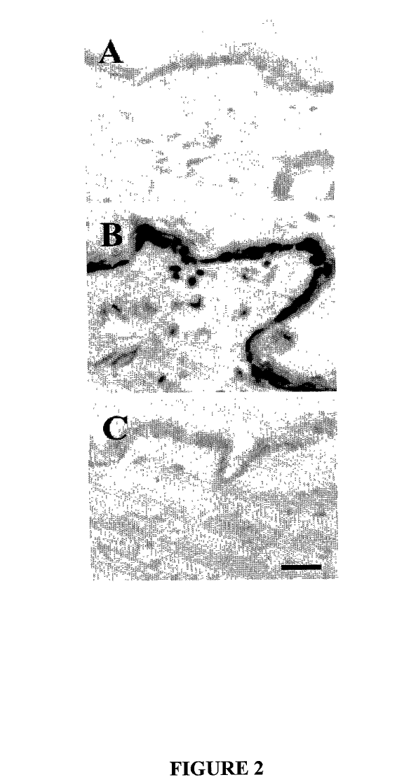

In order to demonstrate the ability of bio-NAD⁺ method to detect PARP activation in tissues, Applicants have applied hydrogen peroxide (250 nM/50 μl) on the skin of mice for 30 min. Skin was excised and immediately frozen in cryoembedding medium to preserve enzyme activity. Frozen sections (10 μm) permeabilized with Triton X-100 were incubated with the bio-NAD⁺ substrate followed by biotin detection with streptavidin-peroxidase. In control (vehicle-treated) skin, no detectable ADP-ribosylation was found (FIG. 2A). Peroxynitrite treatment activated PARP in the skin, as indicated by the appearance of darkly stained cells (FIG. 2B). Staining was nuclear and was most intense in keratinocytes. However, some scattered cells in the dermis also showed nuclear PARP activity (FIG. 2B). The presence of the PARP inhibitor 3-aminobenzamide (5 mM) abolished peroxynitrite-induced bio-ADP ribose incorporation, demonstrating that PARP activation was responsible for the staining (FIG. 2C).

### EXAMPLE 4

### CELISA Method for the Detection of PARP Activation

J774.1 cells were seeded in 96 well plates in RPMI/10% fetal bovine serum. On the next day, cells were treated with the PARP inhibitor PJ34 (5 μM) for 30 min and then stimulated with hydrogen peroxide (100-800 μM). Medium was then replaced by PARP reaction buffer (56 mM HEPES, 28 mM KCl , 28 mM NaCl, 2 mM MgCl₂) containing 0.01% digitonin, 10 μM NAD and 30 μM biotinylated NAD⁺ . Plates were incubated for 30 min at 37° C. Buffer was then aspirated and cells were fixed by the addition of 200 μl/well prechilled 95% ethanol at −20° C. for 10 mm. Endogenous peroxidase activity was blocked by 15 min incubation in 0.5% hydrogen peroxide/methanol. Wells were washed once with 300 μl/well PBS and then blocked by 1% BSA in PBS (200 μl/well) for 30 min at 37° C. BSA solution was then aspirated and replaced by 50 μl/well peroxidase-labeled streptavidine (diluted 1:500 in 1% BSA-PBS). After incubation (30 min at 37° C.), plates were washed three times with PBS and reaction was developed with TACS-Sapphire™ (Trevigen) substrate (100 μl/well). The optical density was measured with a microplate spectrophotometer (Molecular Devices, Sunnyvale, Calif.).

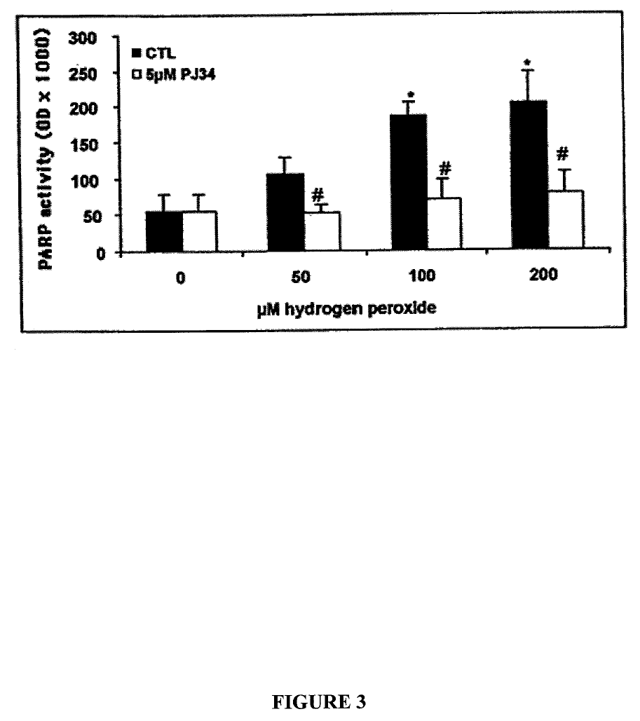

The results are shown in FIG. 3. Results are given as mean ±SD of quadruplicate samples. (* indicates significantly (p<0.05) increased PARP activity as compared to control; # indicates significant (p<0.05) suppression of PARP activity by PJ34.

A cellular ELISA method allows the quantification of PARP activity. Furthermore, the potency of pharmacological PARP inhibitors in cells can also be determined in a CELISA. J774 macrophages seeded in 96 well plates were exposed to hydrogen peroxide (100-800 μM) in the presence or absence of the novel, potent PARP inhibitor PJ34 (5 μM) (Soriano et al., 2001, Nat Med 7:108-13 and Abdelkarim et al., 2001, Int J Mol Med 7:255-260). Hydrogen peroxide induced a dose-dependent PARP activation in J774 cells, and pretreatment with the PARP inhibitor PJ-34 suppressed hydrogen peroxide-induced PARP activation.

What is claimed is:

1. A method for identifying a poly(ADP-ribose) polymerase-expressing cell, said method comprising:
(a) contacting a cell with an effective amount of biotinylated NAD⁺, and
(b) detecting the presence of incorporated biotin within the cell.

2. A method for identifying a poly(ADP-ribose) polymerase-expressing tissue, said method comprising:
(a) contacting a tissue with an effective amount of biotinylated NAD⁺ , and
(b) detecting the presence of incorporated biotin within the tissue.

3. A method for determining the amount of poly(ADP-ribose) polymerase activity in a cell, said method comprising:
(a) contacting a poly(ADP-ribose) polymerase-expressing cell with an effective amount of biotinylated NAD⁺, and
(b) determining the amount of incorporated biotin within the cell.

4. A method for identifying inflamed tissue, said method comprising:
(a) contacting the tissue with an effective amount of biotinylated NAD⁺ , and
(b) detecting the presence of incorporated biotin within the tissue.

5. A method for identifying a patient having an oxidative-stress disorder, comprising:
(a) contacting a cell or tissue of the patient with an effective amount of biotinylated NAD⁺, and
(b) detecting the presence of incorporated biotin within the cell or tissue.

6. A method for identifying a patient having a disorder, wherein the disorder is selected from a group consisting of inflammation, shock, reperfusion injury, stroke, myocardial ischemia, diabetes, diabetic endothelial dysfunction, atherogenesis, and atherosclerosis comprising:
(a) contacting a cell or tissue of the patient with an effective amount of biotinylated NAD⁺, and
(b) detecting the presence of incorporated biotin within the cell or tissue.

7. The method of any one of claims 1 to 6 wherein the contacting is in situ.

8. The method of any one of claims 1 to 6 wherein the contacting is in vivo.

9. The method of any one of claims 1 to 6 wherein incorporated biotin is detected with a conjugated streptavidin detection system.

10. The method of claim 9 wherein the conjugated streptavidin detection system is streptavidin peroxidase or peroxidase-conjugated streptavidin.

11. The method of claim 1, 3, 5 or 6 wherein the method further comprises fixing the cell with trichloracetic acid (TCA).

12. The method of claim 1, 3, 5 or 6 wherein the cell is selected from a group consisting of mitotic cells, oxidatively stressed cells, macrophages, thymocytes, endothelial cells, neuronal cells, and fibroblasts.

13. The method of claim 2 or 4 wherein the tissue comprises cells selected from a group consisting of mitotic cells, oxidatively stressed cells, macrophages, thymocytes, endothelial cells, neuronal cells, and fibroblasts.

14. The method of claim 2 or 4 wherein the tissue is selected from a group consisting of skin and vascular tissue.

15. The method of claim 5 wherein the oxidative-stress disorder is selected from a group consisting of ischemia-reperfusion injury, cancer, aging, arthritis associated with age, fatigue associated with age, alcoholism, red blood cell defects, iron overload, kidney defects, gastrointestinal tract defects, inflammatory-immune injury, brain injury, heart and cardiovascular system defects, eye injury, amyotrophic lateral sclerosis, and age-related macular degeneration.

16. The method of claim 2 or 4 wherein the presence of incorporated biotin within a tissue is determined comprising:
(a) detecting incorporated biotin with a conjugated streptavidin detection system;
(b) developing a reaction of said conjugated streptavidin with said incorporated biotin with a colorimetric substrate; and
(c) counterstaining said incorporated biotin reacted with said calorimetric substrate.

17. The method of claim 16 wherein the streptavidin detection system is streptavidin peroxidase or peroxidase-conjugated streptavidin.

18. The method of claim 16 wherein the colorimetric substrate is cobalt-enhanced nickel-DAB substrate and the counterstain is Nuclear Fast Red.

19. The method of claim 3 wherein the amount of incorporated biotin within a cell is measured comprising:
(a) detecting incorporated biotin with a conjugated streptavidin detection system;
(b) developing a reaction of said conjugated streptavidin with said incorporated biotin with a calorimetric substrate; and
(c) measuring optical density of said calorimetric substrate with a spectrophotometer.

20. The method of claim 19 wherein the streptavidin detection system is streptavidin peroxidase or peroxidase-conjugated streptavidin.
